(19) United States
(12) Patent Application Publication
(10) Pub. No.: US 2007/0059294 A1
(43) Pub. Date: Mar. 15, 2007

(54) Therapeutiv preparation for hematopoietic disease

(76) Inventors: Toshihiro Nakajima, Kanagawa (JP); Tetsuya Amano, Kanagawa (JP); Naoko Yagishita, Kanagawa (JP)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/568,033

(22) PCT Filed: Aug. 13, 2004
(86) PCT No.: PCT/JP04/11951

§ 371 Date: Sep. 25, 2006

Related U.S. Application Data
(60) Provisional application No. 60/495,001, filed on Aug. 13, 2003.

(51) Int. Cl. A61K 48/00; C12N 5/08; A61K 38/18

(52) U.S. Cl. 424/93.21; 514/12; 514/44; 435/455; 435/372

(57) ABSTRACT

The present invention provides therapeutic formulations for hematopoietic diseases comprising as effective ingredient, synoviolin which is reported to be isolated as a synovial cell protein and its gene, and therapeutic methods and such comprising the step of administering the protein or polynucleotide. The present invention also provides as models of hematopoietic diseases, animals whose synoviolin is homozygously deficient and cells derived from such animals; and additionally provides methods that use these models in the screening of therapeutic agents for hematopoietic diseases.

### BACKGROUND

Synoviolin is a membrane protein isolated by immunoscreening using anti-human synovial cell antibodies. This suggests that synoviolin is involved in the onset of rheumatoid arthritis. In fact, although synoviolin is hardly expressed in the synovial tissues of OA patients, strong expression is apparent in the synovial tissues of RA patients. Further, there are reports that mice overexpressing the synoviolin gene develop synovial hyperplasia and rheumatism-like symptoms accompanied by cartilage and bone destruction, and this supports the involvement of synoviolin in RA.

Synoviolin is a human homolog of ubiquitin ligase (E3), which is a yeast Hrd1p with a RING finger motif. This yeast Hrd1p is known as a molecule that eliminates the structurally abnormal proteins produced and accumulated in the endoplasmic reticulum due to stresses such as ischemia, hypoxia, heat shock, amino acid starvation, viral infection, and a reduction in the concentration of endoplasmic reticulum lumenal calcium, and is also known to prevent rupture of the endoplasmic reticulum. There are three endoplasmic reticulum stress response mechanisms: (1) transcriptional induction of folding enzymes and chaperone molecules in the endoplasmic reticulum, (2) suppression of protein translation, and (3) active degradation of abnormal proteins by the ubiquitin-proteasome system. In general, (1) and (2) are referred to as the unfolded protein response (UPR), and (3) is referred to as endoplasmic reticulum-associated degradation (ERAD). Yeast Hrd1p is involved in the ERAD of (3). When subjected to excessive stress beyond the capacity UPR and ERAD, cells choose death (apoptosis). Similarly, ERAD failure also results in the induction of apoptosis.

As described above, synoviolin has E3 activity and is expressed in the ER of cells, and thus, like yeast Hrd1p, synoviolin is expected to be involved in ERAD and to take part in the endoplasmic reticulum stress response mechanism.

Further, past reports suggest that synovial hyperplasia arises from abnormal apoptosis of synovial cells during RA, which allows the inference of a relationship between Synoviolin and apoptosis. Therefore, to clarify these points, we generated Synoviolin gene-deficient mice. Analyses revealed that these mice were embryonic lethal by E13.5 and that apoptosis was increased over their entire body. Further, it was revealed that apoptosis was increased in cells derived from Synoviolin gene-deficient mice as a result of endoplasmic reticulum stress stimuli. These investigations showed that through ERAD, Synoviolin plays a role in avoiding automatic cell death due to endoplasmic reticulum stresses.

### EXPERIMENTAL RESULTS

Generation of Synoviolin KO Mice

To delete the Synoviolin gene, a targeting vector was constructed such that LacZ/Neo genes were inserted in the transcription initiation site (FIG. 1). Since the LacZ gene has an added polyA, the construction of this vector means that the Synoviolin fusion protein is not translated.

First, we attempted to generate homologous KO mice by crossing heterologous mice; however, the birth of homologous Synoviolin KO mice could not be confirmed and they were considered possibly embryonic lethal. Embryos at various developmental stages were analyzed and it was revealed that the embryos had died before E13.5 (FIG. 2).

Thus, Synoviolin was also shown to play an important role in mouse development. A total deficiency of Synoviolin was confirmed at both the transcriptional and translational levels.

Phenotypes of Synoviolin KO Mice

Total imaging and HE staining were performed on E13.5 homologous Synoviolin KO mice. No prominent variations in physical appearance were observed for these homologous Synoviolin KO mice as compared to WT mice and heterologous mice. Further, histological investigations did not reveal any serious variations, such as the deletion of major organs (FIG. 3).

HE Staining of the Liver

HE staining of the liver was performed next. The results of narrow field examination of tissue images confirmed that homologous Synoviolin KO mice had a reduced cell density throughout their entire body as compared to WT mice and +/−mice (FIG.4).

As described above, when homologous Synoviolin mice were generated and their phenotypes were examined, it was revealed that mice were embryonic lethal by E13.5, and that they had reduced cell density throughout their entire body.

KO Mice Which Become Embryonic Lethal Around E13.5

The E13.5 period in mice development, which is when homologous Synoviolin KO mice reach death, is a period of transition between primary hematopoiesis, in which erythroblasts are formed in the umbilical vesicle, and secondary hematopoiesis, in which erythrocytes are formed in the liver of the embryo. Many of the KO mice reported to date also die during this period due to hematopoietic system abnormalities (FIG. 5). To investigate whether homologous Synoviolin KO mice have abnormal hematopoietic systems, peripheral blood from E10.5, which is when primary hematopoiesis occurs, and peripheral blood and liver from E12.5, which is when secondary hematopoiesis occurs, were collected. Samples were prepared by cytospin and then examined.

Giemsa Staining (Primary Hematopoiesis)

First, Giemsa staining was performed using peripheral blood from E10.5, when primary hematopoiesis occurs. At this point, erythroblasts that have differentiated from hematopoietic stem cells can be seen, and erythroblast formation was also observed in homologous Synoviolin KO mice. However, erythroblast formation was generally low in Synoviolin KO mice and images of apoptosis induction were also confirmed (FIG. 6).

Giemsa Staining (Secondary Hematopoiesis)

Next, Giemsa staining was performed using peripheral blood from E12.5, when secondary hematopoiesis occurs. At this point, compared to earlier erythroblasts formed in primary hematopoiesis, erythroblasts are observed in more advanced stages of differentiation, and denucleated mature erythrocytes are also observed. Although mature erythrocytes were present in homologous Synoviolin KO mice, it was revealed that erythroblasts showing such aberrant nucleus patterns had increased: erythroblasts with nuclear disruption, erythroblasts with two-lobed nuclei, and erythroblasts with Howell-Jolly bodies were confirmed to have increased nearly two to three times compared with WT mice (FIG. 7).

Also, the previously described apoptosis of erythroblasts observed during primary hematopoiesis in homologous Synoviolin KO mice was hardly observed at all at this point. The results of Giemsa staining of the liver are a speculative reason for this: images of macrophages taking up erythroblasts, or hemophagocytosis, were hardly observed in WT mice and heterologous mice, but a significant increase of 22 cells out of 100 cells was observed in homologous Synoviolin KO mice (FIG. 7). It is thus predicted that apoptotic erythroblasts, such as those seen in E10.5, are phagocytosed/removed by macrophages in the liver, which is the site of hematopoiesis. As a result, apoptotic erythroblasts are not observed in peripheral blood, as mentioned above.

Hemophagocytosis in the Liver

When reinvestigated in terms of hemophagocytosis, similar phenomena were observed in the liver tissues of homologous Synoviolin KO mice. This supports the previous results observed using Giemsa staining (FIG. 8).

### Conclusions

The results are summarized below: (1) Synoviolin KO mice are embryonic lethal by E13.5; (2) Synoviolin KO mice at E12.5 have more erythroblasts with aberrant morphology and also have enhanced hemophagocytosis; (3) Synoviolin KO mice were revealed to have reduced cell density throughout the body.

From the above insights, obtained from Synoviolin KO mice, the following points were considered regarding the function of Synoviolin: First, Synoviolin somehow regulates apoptosis, and as a consequence, induces aberrant differentiation of erythroblasts. To eliminate the abnormal erythroblasts macrophages are activated, hemophagocytosis is increased, and anemia/lethality due to a reduction in erythrocytes can occur.

On the other hand, it is also concievable that, by the same mechanism as that observed with erythroblast differentiation, lethality occurs after a cell density decrease throughout the body caused by the apoptosis aberration and decreased function in each tissue can occur (FIG. 9).

Thus, we now plan to clarify the detailed molecular mechanisms, focusing on the point of apoptosis enhancement. By clarifying this point, it is presumed that the relationship between Synoviolin and the hematopoietic system, as well as the relationship between Synoviolin and rheumatism, will be clarified. It is further hoped this will lead to the discovery and development of drugs that target their points of action.

### DISCUSSION

Proteins need to take proper conformation in order to exhibit their given functions. Since the endoplasmic reticulum is the place where secreted proteins and transmembrane proteins arrange their conformation, various molecular chaperones and folding enzymes that assist in correct folding of proteins are present in abundance in its lumen and the endoplasmic reticulum functions as an organelle which controls protein quality. When cells receive various physical or chemical stresses from the environment, such as ischemia, hypoxia, heat shock, amino acid starvation, viral infection, or a reduced calcium concentration in the lumen of the endoplasmic reticulum, abnormalities occur in protein folding and proteins with abnormal conformation accumulate in the endoplasmic reticulum. Since this situation is very harmful to the organism, cells respond by inducing transcription of chaperones and folding enzymes in the endoplasmic reticulum, inhibiting protein translation, degrading proteins with abnormal conformation, and so on. Cells also have a crisis management mechanism to solve the accumulation of aberrant proteins in the endoplasmic reticulum to get out of this situation. However, even though cells have such a mechanism, they will choose their own death (apoptosis) when subjected to a stress too serious to be handled, so as not to disturb the harmony of their surroundings. The breakdown of this mechanism similarly results in apoptosis induction.

This kind of research, related to endoplasmic reticulum stress, has so far been conducted mainly in yeast; however, the signal transduction systems related to quality management of proteins in mammalian cells have recently been further clarified, revealing that mammalian cells have developed a more complicated and advanced system than yeast. However, there is still little information regarding endoplasmic reticulum stress at the animal level. Among reports so far,

We generated synoviolin knockout mice lacking synoviolin, which is a protein considered to function as a quality control ligase and participate in ERAD—one of the endoplasmic reticulum stress response mechanisms. They then attempted to elucidate the in vivo mechanism of the protein.

Analyses showed that the synoviolin knockout mice died as fetuses at E12.5-13.5, and increased systemic apoptosis clearly decreased cell density. Surprisingly, deleting the function of synoviolin, which is one of many existing ubiquitin ligases, led to lethality in mice at the developmental stage, which is an absolutely novel and totally unexpected discovery. Further examination of the synoviolin knockout mice showed that these mice have abnormal hematopoietic systems, and hematophagocytosis is increased due to macrophage activation. More specifically, the examination proved that one consequence of augmented apoptosis in synoviolin knockout mice is abnormal erythroblast differentiation, which activates macrophages to eliminate the resulting increase in abnormal erythroblasts. This causes anemia as a result of the reduced number of blood cells, and the mice die at this stage. Examinations using MEFs revealed that, as expected, this augmentation of apoptosis was induced by ER stress. In other words, this result suggests that ER stress strongly affects secondary hematopoiesis during mouse development, leading to the completely novel discovery that failure of ERAD causes lethality in mice. As mentioned earlier, the present study demonstrated for the first time that a factor called synoviolin, one of the ubiquitin ligases, plays a particularly important role in ERAD.

We have previously reported the involvement of synoviolin in the onset of rheumatoid arthritis; therefore, it is thought that therapies for this disease can be provided by targeting synoviolin function, which plays an important role in ERAD. Furthermore, therapeutic effects and such are predicted for not only hematopoietic disorders and rheumatoid arthritis, but also for diseases related to ER stress-induced apoptosis. Thus, the present study is expected to lead to the development of new pharmaceuticals.

Specific Examples of Therapeutic Methods Utilizing Hematopoietic Stem Cells

To obtain hematopoietic cells, multipotent human stem cells must be isolated from the bone marrow, or from other hematopoietic sources. First, bone marrow cells can be obtained from bone marrow sources such as the iliac crest, tibia, femur, spine, or other bone cavities. Other sources for obtaining hematopoietic stem cells include the embryonic umbilical vesicle, fetal liver, fetal and adult spleen, adult peripheral blood, and umbilical cord blood.

Hematopoietic stem cells can be obtained from these tissues by following the procedure of Herenberg, L. A. “Weir's Handbook of Experimental Immunology, 5th edition,” Blackwell Science Inc. 1997. More specifically, cells can be immunologically stained using anti-CD34 antibodies, anti-CD33 antibodies, anti-CD38 antibodies, and such, and then sorted using a cell sorter, based on the stainability of these antibodies.

After hematopoietic stem cells are isolated, they can be grown as follows: Specifically, hematopoietic stem cells can be grown by providing growth factors that are involved in stem cell maintenance by co-culturing these cells with stromal cells obtained from bone marrow cells, fetal thymus, or fetal liver.

For the introduction of therapeutic genes into hematopoietic stem cells or hematopoietic precursor cells, methods generally used for gene introduction into animal cells may be used, including methods that use virus-derived gene therapy vectors for animal cells, such as retrovirus vectors, adenovirus vectors, adeno-associated virus (AAV) vectors, simple herpes virus vectors, and HIV vectors, as well as calcium phosphate co-precipitation methods, DEAE-dextran methods, electroporation methods, liposome methods, lipofection methods, and microinjection methods.

Hematopoietic stem cells into which genes are introduced can be used to treat disorders. By introducing the hematopoietic stem cells obtained by the present invention into the bodies of patients, patients with impaired condition due to reduced formation of the various hemocytes can improve their condition. Otherwise, the vectors for gene introduction generated above can be used to treat disorders by their direct administeration to patients.

### SUMMARY

We recently found that Synoviolin/HRD1 is involved in the pathogenesis of arthropathy. In addition, Synoviolin is implicated in quality control of proteins through the endoplasmic reticulum (ER)-associated degradation (ERAD) system and likely has anti-apoptotic effect in culture cell systems. To understand the role of Synoviolin in vivo, we generated synoviolin-deficient (syno^{-/-}) mice by gene-targeted disruption. Surprisingly, all fetuses lacking syno died in utero by E12.5-13.5. Histologically, syno^{-/-} embryos showed generalized low cellular density and aberrant apoptosis. In hematopoietic system, apoptosis, nuclear fragmentation of erythroblasts, Howell-Jolly body formation and Hemophagocytosis were observed. Apoptosis could be induced through several pathways, such as Fas, irradiation and ER stress. We examined the apoptotic response of cultured embryonic fibroblasts derived form syno^{-/-} mice. These cells were significantly susceptible to ER stress-induced apoptosis but not to Fas and gamma-irradiation. Such susceptibility was rescued by synoviolin-overexpression. Our findings demonstrate the importance of the ERAD system, which is one pathway of ‘quality control of proteins’, in the pathogenesis of arthropathy but also in the normal process of embryo genesis.

### INTRODUCTION

Quality control of proteins in endoplasmic reticulum (ER) and transcriptional control of the amount of proteins in the nucleus are important processes that maintain cellular homeostasis (Hampton, 2002; Shen et al., 2001). In eukaryotic cells, newly synthesized proteins are transported into ER where they are correctly folded. However, various environmental conditions, such as large amount of proteins influx into ER, could trigger a cellular response termed the unfolded protein response (UPR) to overcome this problem (Welihinda et al., 1999). During the UPR response, synthesis of new proteins is globally inhibited by inactivation of eukaryotic initiation factor (eIF) 2α to reduce additional accumulation of misfolded proteins in ER, and genes encoding ER chaperone proteins including Bip/Grp78 and Grp94, are also upregulated to re-fold the misfolded proteins correctly (Ron, 2002). When the amount of misfolded proteins exceeds the protein folding capacity, in spite of UPR, misfolded proteins have to be eliminated by ubiquitin- and proteasome-dependent degradation processes, known as ER-associated degradation (ERAD) (Hampton, 2002). Misfolded proteins in ER are translocated into the cytosol, where they are targeted for 26S proteasome by ubiquitin ligase enzymes. Various ubiquitin ligases are reported in ERAD system in mammalian cells including CHIP (C-terminus of Hsc70-interacting protein) (Ballinger et al., 1999; Meacham et al., 2001; Imai et al., 2002), parkin (Imai et al., 2000), gp78/AMFR (Shimizu et al., 1999; Fang et al., 2001) and Fbx2/FBG1/NFB42 (Yoshida et al., 2002), and intensive research is currently being conducted to determine the precise mechanisms that regulate the ERAD system in ER.

It is reported that the ERAD system constitutively functions to eliminate the amount of misfolded proteins produced during cell growth (Travers et al., 2000). Recent studies have shown that functional disruption of the UPR and/or ERAD system can augment caspase-dependent apoptosis of cells treated with some ER stress inducible chemical agents (Nakagawa et al., 2000), which are known to disturb proper protein folding in ER (Lee, 2001). These results can explain the molecular pathogenesis of certain human diseases that arise from ERAD system disruption. For instance, production of expanded polyglutamine causes certain inherited neurodegenerative disorders (Jana et al., 2001; Bence et al., 2001; Hirabayashi et al., 2001), or mutation in parkin gene, a famous ubiquitin ligase protein in ERAD system, is thought to result in neuronal death of the substantia nigra in patients with autosomal recessive juvenile parkinsonism (AR-JP) (Imai et al., 2000). These findings emphasize the importance of the ERAD system in cell survival in both physiological and pathological conditions.

Recently, by immunoscreening using anti-synovial cell antibodies, we cloned Synoviolin/HRD1, a human homologue of yeast ubiquitin ligase (E3) Hrd1p/De13 (Bays et al., 2001), which is an ER resident membrane protein with RING-H2 motif. This molecule is overexpressed in the rheumatoid synovium, and 10 out of 33 littermates of synoviolin-overexpressing mice developed spontaneous arthropathy. Moreover, in collagen-induced arthritis (CIA) model, only 7% of synoviolin^{+/-} mice developed arthritis compared with 65% of wild-type littermates. In addition, the proportion of cells positive for terminal-deoxynucleotidyl transferase mediated d-UTP nick end labeling (TUNEL) was significantly increased in synovial tissues of synoviolin^{+/-} mice with CIA (Amano et al., submitted manuscript). Recently, Kaneko et al. reported that this protein has anti-apoptotic effects against ER stress-induced apoptosis. These findings can provide a novel model for the pathogenesis of rheumatoid arthritis mediated by the E3 ubiquitin ligase, Synoviolin, through its anti-apoptotic effect on synovial cells.

To define the ‘physiological’ function of Synoviolin in vivo, we have analyzed synoviolin-deficient mice. Our results indicate that Synoviolin plays an indispensable role in the normal process of embryogenesis through ERAD system.

### RESULTS

Targeted Disruption of the Synoviolin/hrd1 Gene Results in Embryonic Lethality

To disrupt the mouse synoviolin/hrd1 gene, we constructed the targeting vector (FIG. 1A). Homologous recombination of this vector into the synoviolin locus inserts a lacZ reporter gene in frame and a neomycin resistance gene at the ATG translational start site. Since the lacZ cassette contains poly(A) addition sequence, it is expected to prevent translation of a Synoviolin fusion protein. After electroporation and drug selection, neo-resistant TT2 embryonic stem clones were isolated and genotyped by Southern blot analysis using a probe described in FIG. 1A. Two independently targeted clones were injected into ICR 8-cell and gave germ line transmission (FIG. 1B). F1 mice heterozygous (syno^{+/-}) for the mutation were viable, fertile, and had no apparent phenotypic abnormalities (data not shown). Heterozygous mice were interbred to generate homozygous mutants (syno^{-/-}), and newborn offspring were genotyped, but no syno^{-/-} mice were identified, indicating that loss of Synoviolin is incompatible with normal embryogenesis (Table 1).

To determine the nature of this targeted Synoviolin mutation, total RNA and protein were prepared from E13.0 embryos and analyzed by Northern and Western blotting, respectively. As shown in FIGS. 1C and D, no Synoviolin mRNA and proteins were detected in the homozygous embryos. Thus, the homozygous mutant embryos expressed neither Synoviolin transcripts nor polypeptides.

Apoptotic Cell Death in syno^{-/-} embryos

To identify the stage of embryonic development at which the synoviolin mutation is lethal, we analyzed E10.5-E18.5 embryos. At E11.5, the majority of synoviolin-deficient embryos (88.9%) were viable; however, by E13.5, very few syno^{-/-} embryos were found alive (Table 1). Morphological analysis of embryos at E13.5 revealed no difference between syno^{-/-} and wild-type littermates (FIG. 2A). However, histological examination of syno^{-/-} embryos showed that the cellular density was obviously decreased in several organs such as the liver (FIG. 2B and C). We assumed that the low cell density was due to augmented apoptotic cell death in syno^{-/-} embryos. To determine the extent of apoptotic cell death induced by loss of Synoviolin, TUNEL was performed at each stage of embryos. At E11.5, apoptotic cells were detected ubiquitously, and the number of apoptotic cells was markedly increased in syno^{-/-} embryos compared with the wild-type counterparts (FIG. 3A). At this stage of embryos, Synoviolin is ubiquitously expressed, including hematopoietic cells (data not shown).

Aberrant Apoptosis in Hematopoiesis of syno^{-/-} embryos

During normal development of murine embryos, the major organ of hematopoiesis shifts at around E11.5-E12.5 from the yolk sac to fetal liver (Zon, 1995). Based on analyses of various genes of the null mutant mouse, several studies have concluded that impairment of hematopoiesis is often associated lethality at this stage (Nuez et al., 1995; Okuda et al., 1996; Kieran et al., 1996; Wang et al., 1996; Tamura et al., 2000; Spyropoulos et al., 2000; Kawane et al., 2001). To investigate the status of hematopoiesis in syno^{-/-} embryos, cytocentrifuge preparations of peripheral blood from E10.5, E12.5, and fetal liver from E12.5 were examined. Staining of peripheral blood samples obtained from E10.5 showed diminished erythroblasts formation in syno^{-/-} embryos (8.3±0.46×10⁵ cells) compared with that of wild-type littermates (3.0±0.66×10⁵ cells, FIG. 4A). Moreover, apoptosis was observed in syno^{-/-} erythroid precursors, whereas it was not observed in wild-type erythroblasts (FIG. 4A, arrowhead). Staining of peripheral blood obtained from E12.5 showed reduced number of erythroblasts in syno^{-/-} embryos (18.0±0.19 ×10⁵ cells) compared with wild-type (4.0±0.18×10⁵ cells). Furthermore, the proportion of abnormal erythroblasts in syno^{-/-} embryos, such as those with Howell-Jolly bodies and nuclear fragmentation, were markedly higher than that of wild-type littermates (10.6% compared with 1.8%) (FIG. 4B, arrowhead). In addition, the percentage of erythroblasts phagocytosed by macrophages was significantly higher in the liver of syno^{-/-} embryos (22.0%) compared with wild-type (1.0%, FIG. 4C, arrowhead). This phenomenon was also observed in histological sections of syno^{-/-} fetal livers (FIG. 2C). These results suggested that macrophages were activated to phagocytose abnormal erythroblasts. Considered together, these findings indicate that lack of Synoviolin is associated with abnormal erythroid differentiation caused by augmentation of apoptosis, and it was likely that syno^{-/-} embryos died of anemia as a result of reduced number of circulating erythroblasts.

syn^{-/-} Mouse Embryonic Fibroblasts are Selectively Susceptible to ER Stress

Apoptosis can be induced through several pathways. To identify the apoptotic pathway that describes Synoviolin involvement, mouse embryonic fibroblasts (MEFs) isolated from syno^{-/-} and wild-type mice were treated in vitro with the following four apoptotic stimuli, monoclonal anti-Fas antibodies (Abs), gamma-irradiation, tunicamycin (N-glycosylation inhibitor), and thapsigargin (Ca²-ATPase inhibitor). Under control conditions, the proportion of syno^{-/-} MEF apoptotic cells was higher (16±4%) than MEF wild-type (6±2%, FIG. 5A). Fas stimulation or exposure to gamma-irradiation did not alter the number of apoptotic syno^{-/-} MEFs and wild-type MEFs (Fas: syno^{-/-,} 45±2%, wild-type, 43±4%; gamma-irradiation: syno^{-/-,} 34±6%, wild-type, 31±2%, FIG. 5A). In contrast, ER stress-inducing agents, tunicamycin and thapsigargin, resulted in 1.7- and 2.4-fold increase in number of TUNEL-positive syno^{-/-} MEFs, respectively, compared with wild-type MEFs (56±3% compared with 33±7%; 90±1% compared with 38±7%, FIG. 5A). Moreover, the sensitivity against ER stress-induced agents was increased in a dose-dependent fashion (FIG. 5B). Furthermore, ER stress-induced apoptosis of syno^{-/-} MEFs was rescued by infection with synoviolin using an adenovirus, although this procedure did not rescue Fas-mediated and gamma-irradiation-induced apoptosis (FIG. 5C). Considered together, these results indicate that Synoviolin rescues cells from ER stress-induced apoptosis of MEFs, but not from Fas- or gamma-irradiation-induced apoptosis. In addition, expression of ER stress inducible proteins, such as CHOP/Gadd153, ATF-6, caspase-12 and so on, were analyzed by western blotting, and induction of these proteins was observed in syno^{-/-} MEFs (data not shown).

### DISCUSSION

The formation of a proper three-dimensional structure is indispensable for protein function. The ‘quality control of proteins’ by UPR and ERAD plays an important role in maintenance of cellular function. Extensive research in recent years has focused on quality control of proteins and the details of signal transduction in vitro have already been identified (Mori, 2000; Hampton, 2002). However, information on the quality control system(s) in vivo is still scarce. With regard to UPR, mice deficient in Perk, an eIF2 α kinase responsible for UPR-induced repression of protein synthesis, are morphologically normal at birth, but subsequently show progressive degeneration of the islets of Langerhans, resulting in loss of insulin-secreting beta cells and development of diabetes mellitus (Zhang et al., 2002). To our knowledge, however, there are no in vivo studies regarding ERAD, thought it is known that breakdown of the ERAD system is associated with the development of various neurodegenerative diseases, such as polyglutamine disease and AR-JP. Thus, there is a need for more in vivo studies in order to elucidate the function of ERAD system. To gain insight into the function of Synoviolin/HRD1, a human homologue of the yeast Hrd1p/Del3 (Bays et al., 2001), which is considered to play a central role among ubiquitin ligases (E3) in the ERAD system, we generated in the present study a mouse deficient in synoviolin using embryonic stem (ES) cells in order to clarify the function of ERAD system in vivo.

Our results showed that syno^{-/-} mice died in utero by E12.5-E13.5 (Table 1), and these embryos showed apparently low cellular density in several organs due to extensive apoptosis (FIGS. 2 and 3). In syno^{-/-} embryos, aberrant apoptosis was also observed in the hematopoietic system (FIG. 4). Moreover, MEFs derived from syno^{-/-} fetuses exhibited in vitro a high and selective susceptibility to ER stress (FIG. 5). Thus, it is conceivable that the apoptotic cell death in syno^{-/-} embryos could be induced by ER stress. The aforementioned changes led a reduction in circulating erythroblasts and ultimately to anemia and death. These changes are similar to other studies, which also indicated that abnormal hematopoiesis could lead to death in utero. In other words, our results indicate that ER stress is taken place in erythropoiesis during embryonic development, and hematopoietic system is susceptible to ER stress. It should be emphasized that it is the first report that breakdown of the ERAD system at this stage could lead to embryonic lethality.

We reported recently that Synoviolin is highly expressed in rheumatoid synovial tissues, and contributes to arthropathy because it induces synovial cell hyperplasia through its anti-apoptotic effects (Amano et al., submitted manuscript). The present study lends support to these early findings and suggests that ER stress could be an important aspect of the pathological process in RA, and that the disease might be caused by abnormalities of the ERAD system. In this regard, although Synoviolin is known to be expressed ubiquitously in vivo (data not shown), it causes a limited and selective pathological process; RA. Interestingly, synoviolin-overexpressing mice, induced by using a β-actin promoter, develop spontaneous arthropathy (Amano et al., submitted manuscript), indicating that the amount of Synoviolin is important for the proper function of this protein. Furthermore, the results also suggest the importance of ER stress/ERAD system even if it does not only depend on the temporal/spatial expression of Synoviolin but also on the temporal/spatial distribution of its substrate(s). In this regard, mutation of Parkin, which is expressed broadly in vivo, causes AR-JP, but such pathology is prescribed by the expression of its substrate, Pael receptor, which is specifically expressed in neuronal cells (Imai et al., 2001; Imai et al., 2002). Extrapolation of the above data to RA argues that the crisis of RA is caused by temporal/spatial expression of substrate(s) of Synoviolin. Nevertheless, we hereby found extensive aberrant apoptosis in syno^{-/-} mouse embryos (FIG. 3). Mdm2, a ubiquitin ligase (E3), controls the on/off switch of the apoptosis signal by establishing p53 ubiquitination and this system is thought to be generally implicated in the regulation of cell fate (Oliner et al., 1993; Honda et al., 2000; Fang et al., 2000; Rodriguez et al., 2000). Therefore, it is conceivable that Synoviolin could also control ER stress-induced apoptosis by ubiquitinating certain broadly expressing-target protein(s). We are currently investigating the nature of such substrate(s) and the mechanisms involved in the regulation of Synoviolin gene expression (Tsuchimochi, unpublished observation). Overall, these studies should identify the mechanisms that control the ERAD system and the effect of the balance of Synoviolin and its substrate(s) on such system.

When the relationship between ‘quality control of proteins’ and ‘maintenance of life’ is discussed, Perk seems to a key molecule of UPR. However, mice deficient in this protein are morphologically normal at birth, per sue, Perk does not influence ‘maintenance of life’ at least during embryogenesis. In contrast, deficiency of synoviolin in mice embryos was lethal. In addition, expression of ER stress inducible proteins, including CHOP/Gadd153 and ATF-6 were induced in syno^{-/-} MEFs. These proteins are considered to be involved in UPR against ER stress. Therefore, in syno^{-/-} MEFs, apoptosis appeared to be induced because of breakdown of ERAD system, but not through UPR system responded to ER stress. Namely, the ERAD system seems to be mostly engaged in the quality control of proteins and it is at the same time indispensable in ‘maintenance of life’ especially during embryonic development. Several ubiquitin ligases (E3) such as CHIP, gp78/AMFR, Parkin and Fbx2/FBG1/NFB42 have been reported to be involved in ERAD system. However, ‘loss-of-function’ of Synoviolin causes absolute lethality during embryonic development without any redundancy. Our study clearly indicates that Synoviolin, particularly among other ubiquitin ligases (E3), plays a pivotal role in the ERAD system.

Finally, Synoviolin has several essential roles both ‘physiologically’ by maintaining life at embryonic development, and ‘pathologically’ by being involved in hyperplasia of synovial cells in RA, through the ERAD system (FIG. 6). In conclusion, our findings clearly indicate the distinct contribution of ERAD system in vivo as well as identify Synoviolin as a novel target for potential therapeutics for various diseases including RA.

### Methods

Generation of synoviolin/hrd1 Deficient Mice

The synoviolin/hrd1 cDNA was cloned from strain-C57BL/6 genomic library. An NcoI site was created at the translation initiation codon of the gene and then the NcoI/BamHI fragment of the gene was replaced with the LacZ cassette (Saga et al., 1992). The neomycin phosphotransferase (Neo) gene cassette derived from pMC1neo (Strategene, La Jolla, Calif.) was placed downstream of the LacZ gene. The 1.85-kb EcoRI/NcoI fragment and the 8.5-kb SalI/XhoI fragment of the synoviolin gene were included upstream and downstream of these cassettes, respectively. The negative selection with the DT-A cassette is described previously (Yagi et al., 1993a). The TT2 ES cells, derived from an F1 embryo between C57BL/6 and CBA mice, were grown on embryonic fibroblast feeder cells as described previously (Yagi et al., 1993b). Homologous recombination was checked by Southern blot analysis. Two independent synoviolin^{+/-} ES clones were injected into ICR 8-cell embryos (Yagi et al., 1993a). Chimeric males with greater than 80% agouti coat color were bred into C57BL/6 or DBA1 females, and germ line transmission of the mutant allele was identified by Southern blot analysis or by PCR analysis of tail- and yolk sac-derived genomic DNA. The following primers were used for genotyping of embryos; 5′-ACACAGTCACCTCCGGTTCTGTA TTCACTG-3′ (P1) and 5′-CTCAGTAACAGCGTACCAGGACCGTTCCAG-3′ (P2). PCR analysis using these primers in 1 cycle at 9° C. for 1 min followed by 35 cycles at 98° C. for 20 s, 68° C. for 10 min, with an extension step of 10 min at 72° C. at the end of last cycle, produced 6.9 kb and 2.6 kb fragments from the mutant an wild alleles, respectively.

RNA Isolation and Northern Blot Analysis

Total RNA was isolated from E13.0 embryos using Isogen (Nippon Gene) based on the acid guanidinium thiocyanate phenol chloroform extraction method (Chomczynski et al., 1987). Then, 20 μg of total RNA were denatured with glyoxal, separated by electrophoresis, and transferred onto a nylon membrane. The membrane was hybridized with a DNA probe corresponding to bases 1234-3028 of the cloned synoviolin cDNA fragment.

Isolation and Histological Analysis of Embryo

Embryos were removed from the uterus, and yolk sac was taken for genotyping. Embryos were then fixed overnight in 4% paraformaldehyde in phosphate-buffered saline (PBS) and embedded in paraffin, and 4-μm sections were cut and stained with hematoxylin and eosin, and some sections were used for the TUNEL assay. Peripheral blood was collected in PBS containing 50% FCS and in 10 mM EDTA for cytospin preparation. Fetal livers from E12.5 embryos were disrupted with a 25-gauge needle in 1 mL of same medium, and approximately 7μL was diluted into 200 μL of medium, and cytocentrifuged. Slides of peripheral blood and fetal liver were stained with May-Grünwald-Giemsa.

TUNEL Assay

Tissues were prepared as described above. Conditioned culture cells were fixed in 10% formalin for 15 minutes and attached to APS-coated slide glasses. These specimens were subjected to TUNEL assay. TUNEL assay was performed according to the protocol provided by the manufacturer (Apoptosis in situ detection kit; Wako, Osaka, Japan).

Isolation of MEFs

MEFs were isolated from E10.5 embryos of syno^{-/-} and wild-type mice by trypsin digestion after removal of the head and internal organs. Isolated cells were cultured in Dulbecco's modified Eagle's medium containing 20% fetal calf serum (FCS).

X-Irradiation

X-irradiation was performed by using MBR-1505R2 X-ray generator (Hitachi Medical, Co., Japan) at 150 kV and 15 mA with a 1.00 mm-A1 filter at a dose rate of 1.04 Gr/min. Total X-ray irradiation was 6Gy.

Adenovirus Infection

Adenovirus vectors containing genes for Flag-taged Synoviolin or LacZ were prepared by Adeno-X™ Expression System according to manufacture's instruction (Clontech Laboratories, Inc., Polo Alto, Calif.). The viral preparations were titrated with end-point dilution assay on HEK293 cells. The number of virus particles (measured in plaque forming units: PFU) per cell was expressed as moi. Infected MEFs were allowed to express targeted genes for 48 hours, then treated with indicated reagents.

Statistical Analysis

Differences between groups were examined for statistical significance using the Student's t-test. A P value less than 0.05 denoted the presence of a statistically significant difference.

Ethical Considerations

All experimental protocols described in this study were approved by the Ethics Review Committees of St. Marianna University School of Medicine and a signed consent form was obtained from each subject participating in our study.

### REFERENCES

Ballinger, C. A., Connell, P., Wu, Y., Hu, Z., Thompson, L. J., Yin, L. Y., and Patterson, C. 1999. Identification of CHIP, a novel tetratricopeptide repeat-containing protein that interacts with heat shock proteins and negatively regulates chaperone functions. Mol. Cell Biol. 19: 4535-4545.
Bays, N. W., Gardner, R. G, Seelig, L. P., Joazeiro, C. A., and Hampton, R. Y. 2001. Hrd1p/Der3p is a membrane-anchored ubiquitin ligase required for ER-associated degradation. Nat. Cell Biol. 3: 24-29.
Bence, N. F., Sampat, R. M., and Kopito, R. R. 2001. Impairment of the ubiquitin-proteasome system by protein aggregation. Science 292: 1552-1555.
Chomczynski, P., and Sacchi, N. 1987. Single-step method of RNA isolation by acid guanidinium thiocyanate-phenol-chloroform extraction. Anal. Biochem. 162: 156-159.
Fang, S., Ferrone, M., Yang, C., Jensen, J. P., Tiwari, S., and Weissman, A. M. 2001. The tumor autocrine motility factor receptor, gp78, is a ubiquitin protein ligase implicated in degradation from the endoplasmic reticulum. Proc. Natl. Acad. Sci. U.S.A. 98: 14422-14427.
Fang, S., Jensen, J. P., Ludwig, R. L., Vousden, K. H., and Weissman, A. M. 2000. Mdm2 is a RING finger-dependent ubiquitin protein ligase for itself and p53. J Biol. Chem. 275: 8945-8951.
Hampton, R. Y 2002. ER-associated degradation in protein quality control and cellular regulation. Curr. Opin. Cell Biol. 14: 476-482.
Hirabayashi, M., Inoue, K., Tanaka, K., Nakadate, K., Ohsawa, Y, Kamei, Y, Popiel, A. H., Sinohara, A., Iwamatsu, A., Kimura, Y., Uchiyama, Y., Hori, S., and Kakizuka, A. 2001. VCP/p97 in abnormal protein aggregates, cytoplasmic vacuoles, and cell death, phenotypes relevant to neurodegeneration. Cell Death. Differ. 8: 977-984.
Honda, R., and Yasuda, H. 2000. Activity of MDM2, a ubiquitin ligase, toward p53 or itself is dependent on the RING finger domain of the ligase. Oncogene 19: 1473-1476.
Imai, Y., Soda, M., Hatakeyama, S., Akagi, T., Hashikawa, T., Nakayama, K. I., and Takahashi, R. 2002. CHIP is associated with Parkin, a gene responsible for familial Parkinson's disease, and enhances its ubiquitin ligase activity. Mol. Cell 10: 55-67.
Imai, Y., Soda, M., and Takahashi, R. 2000. Parkin suppresses unfolded protein stress-induced cell death through its E3 ubiquitin-protein ligase activity. J. Biol. Chem. 275: 35661-35664.
Imai, Y., Soda, M., Inoue, H., Hattori, N., Mizuno, Y., and Takahashi, R. 2001. An Unfolded Putative Transmembrane Polypeptide, which Can Lead Endoplasmic Reticulum Stress, Is a Substrate of Parkin. Cell 105: 891-902
Jana, N. R., Zemskov, E. A., Wang, G. h., and Nukina, N. 2001. Altered proteasomal function due to the expression of polyglutamine-expanded truncated N-terminal huntingtin induces apoptosis by caspase activation through mitochondrial cytochrome c release. Hum. Mol. Genet. 10: 1049-1059.
Kaneko, M., Ishiguro, M., Niinuma, Y, Uesugi, M., and Nomura, Y. 2002. Human HRD1 protects against ER stress-induced apoptosis through ER-associated degradation. FEBS Lett. 532: 147-152.
Kawane, K., Fukuyama, H., Kondoh, G, Takeda, J., Ohsawa, Y, Uchiyama, Y., and Nagata, S. 2001. Requirement of DNase II for definitive erythropoiesis in the mouse fetal liver. Science 292: 1546-1549.
Kieran, M. W., Perkins, A. C., Orkin, S. H., and Zon, L. I. 1996. Thrombopoietin rescues in vitro erythroid colony formation from mouse embryos lacking the erythropoietin receptor. Proc. Natl. Acad. Sci. U.S.A. 93: 9126-9131.
Lee, A. S. 2001. The glucose-regulated proteins: stress induction and clinical applications. Trends Biochem. Sci. 26: 504-510.
Meacham, G. C., Patterson, C., Zhang, W., Younger, J. M., and Cyr, D. M. 2001. The Hsc70 co-chaperone CHIP targets immature CFTR for proteasomal degradation. Nat. Cell Biol. 3: 100-105.
Nakagawa, T., Zhu, H., Morishima, N., Li, E., Xu, J., Yankner, B. A., and Yuan, J. 2000. Caspase-12 mediates endoplasmic-reticulum-specific apoptosis and cytotoxicity by amyloid-beta. Nature 403: 98-103.
Nuez, B., Michalovich, D., Bygrave, A., Ploemacher, R., and Grosveld, F. 1995. Defective haematopoiesis in fetal liver resulting from inactivation of the EKLF gene. Nature 375: 316-318.
Okuda, T., vanDeursen, J., Hiebert, S. W., Grosveld, G, and Downing, J. R. 1996. AML1, the target of multiple chromosomal translocations in human leukemia, is essential for normal fetal liver hematopoiesis. Cell 84: 321-330.
Oliner, J. D., Pietenpol, J. A., Thiagalingam, S., Gyuris, J., Kinzler, K. W., and Vogelstein, B. 1993. Oncoprotein MDM2 conceals the activation domain of tumour suppressor p53. Nature 362: 857-860.
Rodriguez, M. S., Desterro, J. M., Lain, S., Lane, D. P., and Hay, R. T. 2000. Multiple C-terminal lysine residues target p53 for ubiquitin-proteasome-mediated degradation. Mol. Cell Biol. 20: 8458-8467.
Ron, D. 2002. Translational control in the endoplasmic reticulum stress response. J. Clin. Invest. 110: 1383-1388.
Saga, Y., Yagi, T., Ikawa, Y, Sakakura, T., and Aizawa, S. 1992. Mice develop normally without tenascin. Genes Dev. 6: 1821-1831.
Shen, X., Ellis, R. E., Lee, K., Liu, C. Y., Yang, K., Solomon, A., Yoshida, H., Morimoto, R., Kurnit, D. M., Mori, K., and Kaufman, R. J. 2001. Complementary signaling pathways regulate the unfolded protein response and are required for C. elegans development. Cell 107: 893-903.
Shimizu, K., Tani, M., Watanabe, H., Nagamachi, Y., Niinaka, Y., Shiroishi, T., Ohwada, S., Raz, A., and Yokota, J. 1999. The autocrine motility factor receptor gene encodes a novel type of seven transmembrane protein. FEBS Lett. 456: 295-300.
Spyropoulos, D. D., Pharr, P. N., Lavenburg, K. R., Jackers, P., Papas, T. S., Ogawa, M., and Watson, D. K. 2000. Hemorrhage, impaired hematopoiesis, and lethality in mouse embryos carrying a targeted disruption of the Fli1 transcription factor. Mol. Cell Biol. 20: 5643-5652.
Swanson, R., Locher, M., and Hochstrasser, M. 2001. A conserved ubiquitin ligase of the nuclear envelope/endoplasmic reticulum that functions in both ER-associated and Matalpha2 repressor degradation. Genes Dev. 15: 2660-2674.
Tamura, K., Sudo, T., Senftleben, U., Dadak, A. M., Johnson, R., and Karin, M. 2000. Requirement for p38alpha in erythropoietin expression: a role for stress kinases in erythropoiesis. Cell 102: 221-231.
Travers, K. J., Patil, C. K., Wodicka, L., Lockhart, D. J., Weissman, J. S., and Walter, P. 2000. Functional and genomic analyses reveal an essential coordination between the unfolded protein response and ER-associated degradation. Cell 101: 249-258.
Wang, Q., Stacy, T., Miller, J. D., Lewis, A. F., Gu, T. L., Huang, X., Bushweller, J. H., Bories, J. C., Alt, F. W., Ryan, G., Liu, P. P., Wynshaw-Boris, A., Binder, M., Marin-Padilla, M., Sharpe, A. H., and Speck, N. A. 1996. The CBFbeta subunit is essential for CBFalpha2 (AML1) function in vivo. Cell 87: 697-708.
Welihinda, A. A., Tirasophon, W., and Kaufman, R. J. 1999. The cellular response to protein misfolding in the endoplasmic reticulum. Gene Expr. 7: 293-300.
Yagi, T., Nada, S., Watanabe, N., Tamemoto, H., Kohmura, N., Ikawa, Y., and Aizawa, S. 1993. A novel negative selection for homologous recombinants using diphtheria toxin A fragment gene. Anal. Biochem. 214: 77-86.
Yagi, T., Tokunaga, T., Furuta, Y., Nada, S., Yoshida, M., Tsukada, T., Saga, Y., Takeda, N., Ikawa, Y., and Aizawa, S. 1993. A novel ES cell line, TT2, with high germline-differentiating potency. Anal. Biochem. 214: 70-76.
Yoshida, Y., Chiba, T., Tokunaga, F., Kawasaki, H., Iwai, K., Suzuki, T., Ito, Y, Matsuoka, K., Yoshida, M., Tanaka, K., and Tai, T. 2002. E3 ubiquitin ligase that recognizes sugar chains. Nature 418: 438-442.
Zhang, P., McGrath, B. C., Reinert, J., Olsen, D. S., Lei, L., Gill, S., Wek, S. A., Vattem, K. M., Wek, R. C., Kimball, S. R., Jefferson, L. S., and Cavener, D. R. 2002. The GCN2 eIF2alpha kinase is required for adaptation to amino acid deprivation in mice. Mol. Cell Biol. 22: 6681-6688.
Zon, L. I. 1995. Developmental biology of hematopoiesis. Blood 86: 2876-2891.

### FIGURE LEGENDS

FIG. 1. Targeted disruption of the synoviolin/hrd1 gene

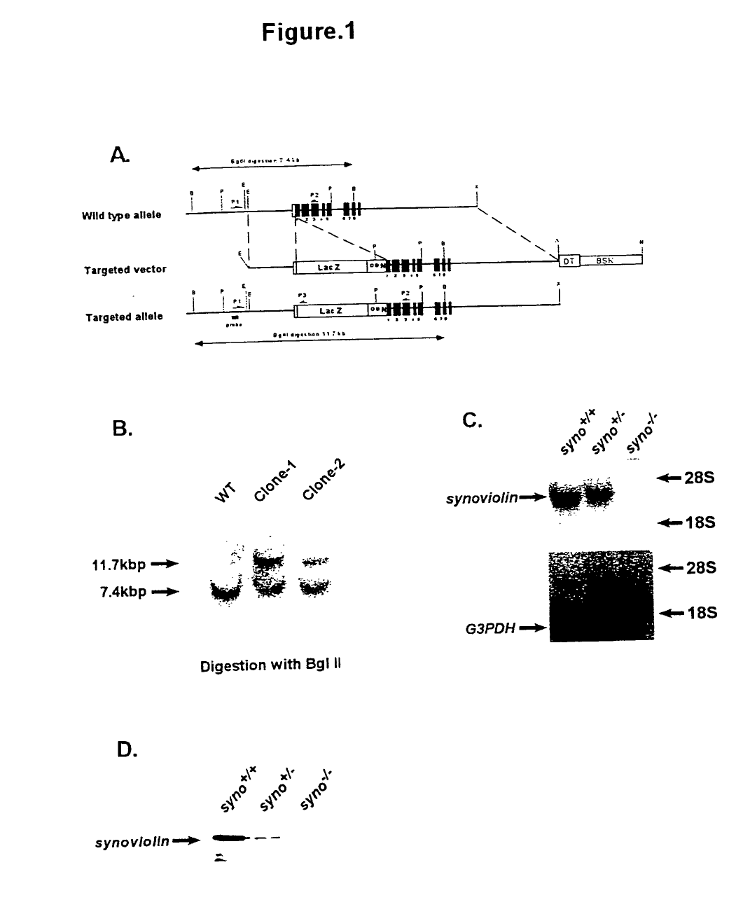

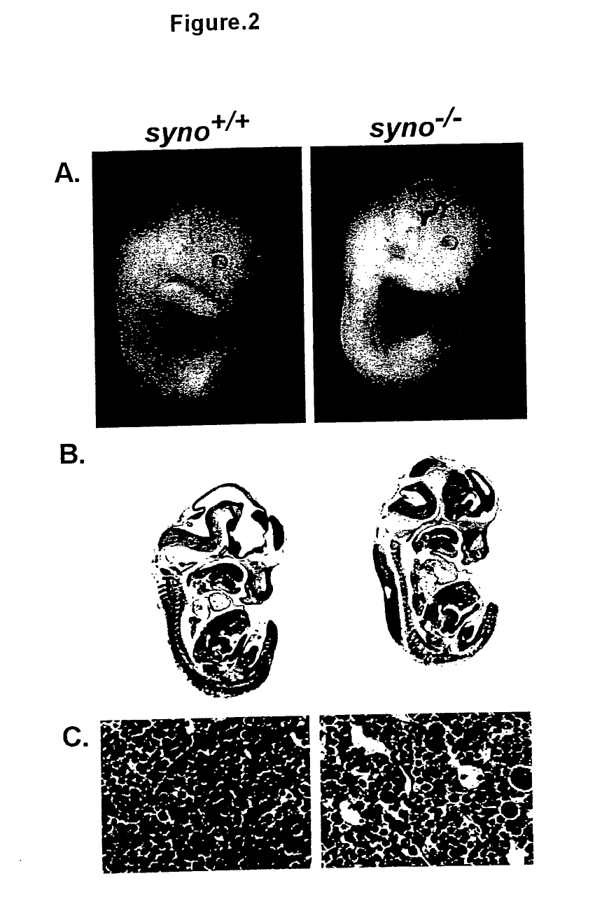

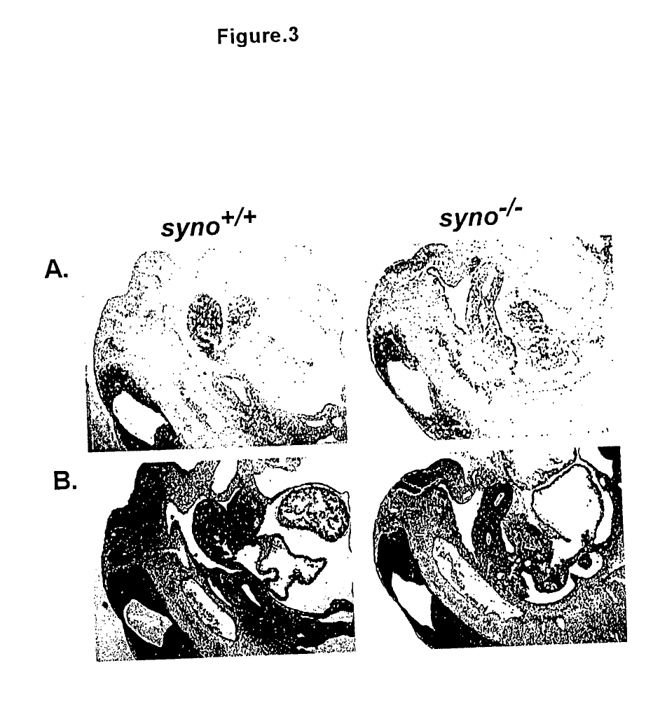

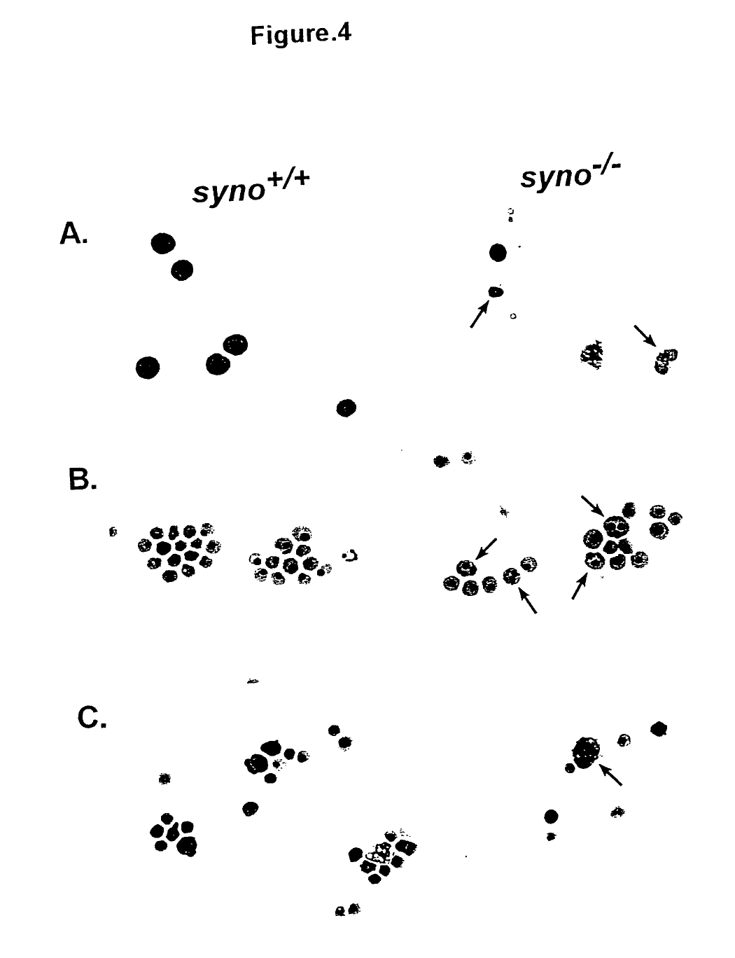

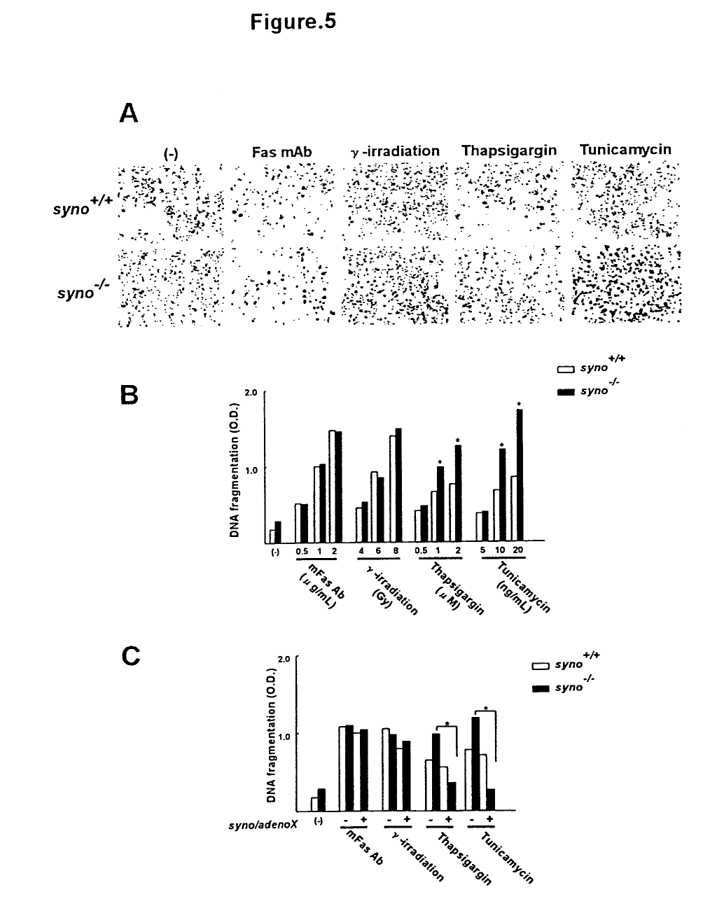

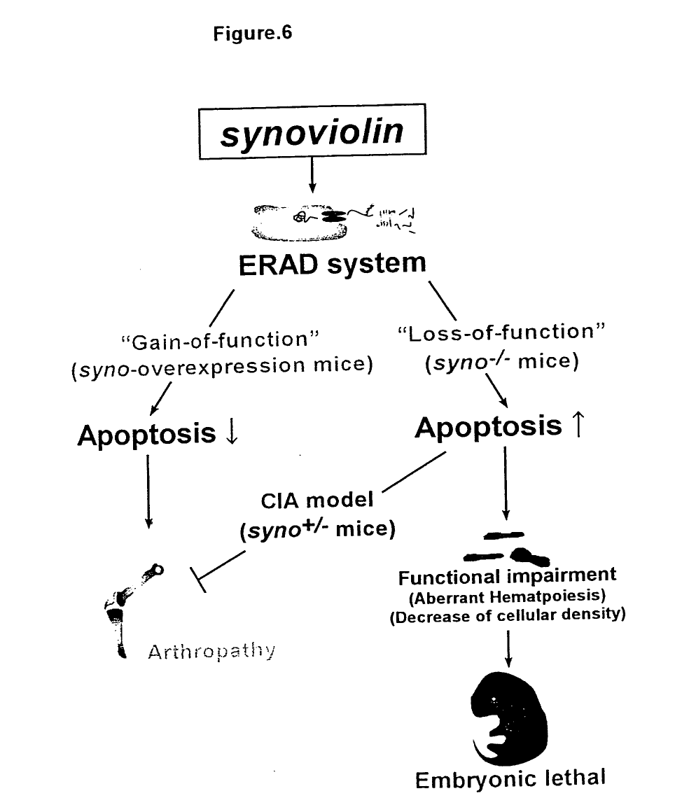

(A) Structure of the synoviolin/hrd1 wild-type allele, the targeting vector, targeted allele, and partial restriction map of the genes before and after targeting events. Exons of the gene are shown as closed boxes, and the β-galactosidase gene (LacZ), neomycin phosphotransferase gene (Neo), diphtheria toxin-A gene (DT), and pBluescript II (BSK) are shown as open boxes. The restriction sites used are indicated: B, BglII; P, PstI; E, EcoRI; X, XhoI; N, NcoI. (B) Southern blot analysis of targeted ES clones. Genomic DNA from wild-type TT2 ES cells (WT) and homologous targeted clones (clone-1, clone-2) were digested with BglII and probed with an external probe. Wild-type and mutant loci generated 7.4-kb and 11.7-kb fragments, respectively. (C) Northern blot analysis. Twenty μg of total RNA, isolated from E13.0 embryos generated by intercrosses used syno^{+/-} mice, hybridized with the probe for synoviolin or glyceraldehyde-3-phosphate dehydrogenase (G3PDH) probe. (D) Total protein was isolated from E13.0 embryos and separated by SDS-PAGE (50 μg protein/lane). After transfer of the protein, the membrane was probed with anti-Synoviolin antibody.

FIG. 2. Phenotypes of E13.5 syno^{-/-} embryos

(A) Appearance of syno^{+/+} and syno^{-/-} E13.5 mouse embryos. The mutant embryo is equal in size compared with the syno^{+/+} and shows normal development. (B) Sagittal sections from syno^{+/+} and syno^{-/-} E13.5 embryos stained with hematoxylin and eosin (×10). (C) Details of liver sections from syno^{+/+} and syno^{-/-} E13.5 (×400). Note the low cell density in syno^{-/-} embryos.

FIG. 3. Apoptosis in E11.5 syno^{-/-} embryos

(A) TUNEL assay on the syno^{+/+} and syno^{+/+} embryos. The number of TUNEL-positive cells was higher in syno^{-/-} embryos than in syno^{+/+} embryos. This difference was also observed throughout the entire body of syno^{-/-} embryos (×40). (B) Hematoxylin and eosin staining of syno^{-/-} embryos (×40).

FIG. 4. Hematopoiesis in E10.5 and E12.5 syno^{-/-} embryos

(A) Cytocentrifuge preparation of peripheral blood isolated from E10.5 syno^{+/+} and syno^{-/-} embryos, stained with May-Grünwald-Giemsa. syno^{-/-} embryos showed reduced erythroblast formation and enhanced apoptosis (arrowhead). (B) Cytocentrifuge preparation of peripheral blood from viable E12.5 embryos. Abnormal erythroblasts, nuclear fragmentation and Howell-Jolly bodies are increased in syno^{-/-} embryos compared with syno^{+/+} embryos (arrowhead). (C) Liver cytocentrifuge preparations from E12.5 syno^{+/+} and syno^{-/-} embryos, stained with May-Grunwald-Giemsa. Hemophagocytosis is apparently increased in syno^{-/-} embryos (arrowhead).

FIG. 5. syno^{-/-} MEFs show increased susceptibility to ER stress.

(A) MEFs derived from syno^{+/+} and syno^{-/-} mice were treated with various apoptotic stimuli, including anti-Fas monoclonal antibodies (1μg/ml for 48 hours), X-irradiation (6 Gy for 72 hours), thapsigargin (1 μM for 48 hours) and tunicamycin (10 ng/ml for 48 hours), or untreated (−), in low serum (1%FCS) medium. Apoptotic cells were detected by TUNEL analysis. (B) syno^{+/+} and syno^{-/-} MEFs were treated with indicated dose of stimuli. Apoptosis was measured by quantitation of DNA fragmentation using the cell death detection ELISA method (Boehringer Mannheim). *p<0.01. The number of apoptotic syno^{-/-} MEFs was higher in cultures treated with thapsigargin and tunicamycin compared with the respective syno^{+/+} MEFs. (C) syno^{+/+} and syno^{-/-} MEFs were infected with adenovirus vector (100 moi) carrying LacZ(−) or synoviolin gene(+), then treated with the same aforementioned agents. In syno^{-/-} MEFs, ER-stress induced apoptosis was rescued by infection with synoviolin.

FIG. 6. Synoviolin acts as an anti-apoptotic protein through ERAD system

‘Loss-of-function’ of Synoviolin results in generalized apoptosis through a breakdown of the ERAD system. This process results in reduced cellular density and abnormal hematopoiesis, and consequently causes death of the embryo. On the other hand, ‘gain-of-function’ of Synoviolin results in the development of spontaneous arthropathy through the anti-apoptotic effects of Synoviolin. Furthermore, syno^{+/-} mice are resistant to collagen-induced arthritis (CIA), which is a model frequently used in experiments related to arthritis. Taken together, Synoviolin plays an important role in ‘maintenance of cellular function’ through ERAD system.

1. A method for treating hematopoietic diseases, wherein the method comprises administering any one of the proteins selected from the group consisting of (a) to (d) shown below, or a polynucleotide encoding the protein:
(a) a protein comprising the amino acid sequence of SEQ ID NO: 2;
(b) a protein comprising an amino acid sequence with one or more amino acid substitutions, deletions, insertions, and/or additions in the amino acid sequence of SEQ ID NO: 2, wherein the protein is functionally equivalent to a protein comprising the amino acid sequence of SEQ ID NO: 2;
(c) a protein encoded by a polynucleotide that hybridizes under stringent conditions with a polynucleotide comprising the nucleotide sequence of SEQ ID NO: 1, wherein the protein is functionally equivalent to a protein comprising the amino acid sequence of SEQ ID NO: 2; and
(d) a protein encoded by a polynucleotide comprising a nucleotide sequence with at least 70% or higher homology to the nucleotide sequence of SEQ ID NO: 1, wherein the protein is functionally equivalent to a protein comprising the amino acid sequence of SEQ ID NO: 2.

2. The method of claim 1, wherein the hematopoietic diseases are diseases caused by abnormal erythroblast differentiation.

3. The method of claim 1, which comprises introducing into hematopoietic stem cells a vector harboring the polynucleotide in an expressible state.

4. A method for inducing erythroblast differentiation, wherein the method comprises expressing in hematopoietic stem cells any one of the proteins selected from the group consisting of (a) to (d) shown below:
(a) a protein comprising the amino acid sequence of SEQ ID NO: 2;
(b) a protein comprising an amino acid sequence with one or more amino acid substitutions, deletions, insertions, and/or additions in the amino acid sequence of SEQ ID NO: 2, wherein the protein is functionally equivalent to a protein comprising the amino acid sequence of SEQ ID NO: 2;
(c) a protein encoded by a polynucleotide that hybridizes under stringent conditions with a polynucleotide comprising the nucleotide sequence of SEQ ID NO: 1, wherein the protein is functionally equivalent to a protein comprising the amino acid sequence of SEQ ID NO: 2; and
(d) a protein encoded by a polynucleotide comprising a nucleotide sequence with at least 70% or higher homology to the nucleotide sequence of SEQ ID NO: 1, wherein the protein is functionally equivalent to a protein comprising the amino acid sequence of SEQ ID NO: 2.

5. A pharmaceutical formulation for treating hematopoietic diseases, wherein the formulation comprises as an effective ingredient any one of the proteins selected from the group consisting of (a) to (d) shown below, or a polynucleotide encoding the protein:
(a) a protein comprising the amino acid sequence of SEQ ID NO: 2;
(b) a protein comprising an amino acid sequence with one or more amino acid substitutions, deletions, insertions, and/or additions in the amino acid sequence of SEQ ID NO: 2, wherein the protein is functionally equivalent to a protein comprising the amino acid sequence of SEQ ID NO: 2;
(c) a protein encoded by a polynucleotide that hybridizes under stringent conditions with a polynucleotide comprising the nucleotide sequence of SEQ ID NO: 1, wherein the protein is functionally equivalent to a protein comprising the amino acid sequence of SEQ ID NO: 2; and
(d) a protein encoded by a polynucleotide comprising a nucleotide sequence with at least 70% or higher homology to the nucleotide sequence of SEQ ID NO: 1, wherein the protein is functionally equivalent to a protein comprising the amino acid sequence of SEQ ID NO: 2.

6. The pharmaceutical formulation of claim 5, which is a hematopoietic stem cell harboring the polynucleotide in an expressible state.

7. A method for treating hematopoietic diseases, wherein the method comprises administering an agent that enhances the activity of a protein comprising the amino acid sequence of SEQ ID NO: 2.

8. A therapeutic agent for hematopoietic diseases, which comprises an agent that enhances the activity of a protein comprising the amino acid sequence of SEQ ID NO: 2 as an effective ingredient.

9. A non-human hematopoietic disease model animal embryo whose synoviolin gene function is defective.

10. The non-human hematopoietic disease model animal embryo of claim 9, wherein the non-human animal is a rodent.

11. The non-human hematopoietic disease model animal embryo of claim 10, wherein the rodent is a mouse.

12. The non-human hematopoietic disease model animal embryo of claim 11, which is an embryo of E13.5 or younger.

13. The non-human hematopoietic disease model animal embryo of claim 9, wherein the hematopoietic disease is a disease caused by abnormal erythroblast differentiation.

14. A hematopoietic disease model cell derived from a non-human hematopoietic disease model animal embryo whose synoviolin gene function is defective.

15. The hematopoietic disease model cell of claim 14, which is a fibroblast.

16. A method of screening for therapeutic agents for hematopoietic diseases, wherein the method comprises administering a test substance to a non-human hematopoietic disease model animal embryo whose synoviolin gene function is defective, and evaluating the condition of erythroblasts in the non-human animal embryo.

17. A method of screening for therapeutic agents for hematopoietic diseases, wherein the method comprises the steps of:
(a) administering or contacting an ER stress-inducing agent with a non-human hematopoietic disease model animal embryos whose synoviolin gene function is defective, or a cell derived from the embryos;
(b) administering or contacting a test substance to the hematopoietic disease model non-human animal embryos whose synoviolin gene function is defective, or cells derived from the embryos, before, after, or simultaneously with step (a); and
(c) determining cells in which apoptosis was induced from among the non-human animal embryos or the cells derived from the embryos.

18. A method of screening for ER stress-removing agents, wherein the method includes the steps of:
(a) acting an ER stress-inducing agent on cells whose endogenous synoviolin gene function is defective;
(b) contacting a test substance to the cells before, after, or simultaneously with step (a); and
(c) determining cells in which apoptosis was induced from among the cells.

19. The hematopoietic disease model cell, according to claim 19, wherein the non-human animal is a rodent.

20. The hematopoietic disease model cell, according to claim 14, wherein the rodent is a mouse.

21. The hematopoietic disease model cell, according to claim 14, wherein the embryo is E13.5 or younger.

22. The hematopoietic disease model cell, according to claim 14, wherein the hematopoietic disease is a disease caused by abnormal erythroblast differentiation.


SEQUENCE LISTING (4 sequences)

SEQ ID NO: 1 - DNA, 3374 bp, Homo sapiens
  CDS (403)..(2256)
gccctttcttatgagcatgcctgtgttgggttgacagtgagggtaataatgacttgttggttgattgtagatatagggctctcccttgcaaggtaattaggctccttaaattacctgtaagattttcttgccacagcatccattctggttaggctggtgatcttctgagtagtgatagattggttggtggtgaggtttacaggtgttcccttctcttactcctggtgttggctacaatcaggtggcgtctagagcagcatgggacaggtgggtaaggggagtcttctcattatgcagaagtgatcaacttaaatctctgtcagatctacctttatgtagcccggcagtcgcgcggattgagcgggctcgcggcgctgggttcctggtctccgggccagggcaatgttccgcacggcagtgatgatggcggccagcctggcgctgaccggggctgtggtggctcacgcctactacctcaaacaccagttctaccccactgtggtgtacctgaccaagtccagccccagcatggcagtcctgtacatccaggcctttgtccttgtcttccttctgggcaaggtgatgggcaaggtgttctttgggcaactgagggcagcagagatggagcaccttctggaacgttcctggtacgccgtcacagagacttgtctggccttcaccgtttttcgggatgacttcagcccccgctttgttgcactcttcactcttcttctcttcctcaaatgtttccactggctggctgaggaccgtgtggactttatggaacgcagccccaacatctcctggctctttcactgccgcattgtctctcttatgttcctcctgggcatcctggacttcctcttcgtcagccacgcctatcacagcatcctgacccgtggggcctctgtgcagctggtgtttggctttgagtatgccatcctgatgacgatggtgctcaccatcttcatcaagtatgtgctgcactccgtggacctccagagtgagaacccctgggacaacaaggctgtgtacatgctctacacagagctgtttacaggcttcatcaaggttctgctgtacatggccttcatgaccatcatgatcaaggtgcacaccttcccactctttgccatccggcccatgtacctggccatgagacagttcaagaaagctgtgacagatgccatcatgtctcgccgagccatccgcaacatgaacaccctgtatccagatgccaccccagaggagctccaggcaatggacaatgtctgcatcatctgccgagaagagatggtgactggtgccaagagactgccctgcaaccacattttccataccagctgcctgcgctcctggttccagcggcagcagacctgccccacctgccgtatggatgtccttcgtgcatcgctgccagcgcagtcaccaccacccccggagcctgcggatcaggggccaccccctgccccccaccccccaccactcttgcctcagccccccaacttcccccagggcctcctgcctccttttcctccaggcatgttcccactgtggccccccatgggcccctttccacctgtcccgcctccccccagctcaggagaggctgtggctcctccatccaccagtgcagcagccctttctcggcccagtggagcagctacaaccacagctgctggcaccagtgctactgctgcttctgccacagcatctggcccaggctctggctctgccccagaggctggccctgcccctggtttccccttccctcctccctggatgggtatgcccctgcctccaccctttgccttccccccaatgcctgtgccccctgcgggctttgctgggctgaccccagaggagctacgagctctggagggccatgagcggcagcacctggaggcccggctgcagagcctgcgtaacatccacacactgctggacgccgccatgctgcagatcaaccagtacctcaccgtgctggcctccttggggcccccccggcctgccacttcagtcaactccactgaggggactgccactacagttgttgctgctgcctcctccaccagcatccctagctcagaggccacgaccccaaccccaggagcctccccaccagcccctgaaatggaaaggcctccagctcctgagtcagtgggcacagaggagatgcctgaggatggagagcccgatgcagcagagctccgccggcgccgcctgcagaagctggagtctcctgttgcccactgacactgccccagcccagccccagcctctgctcttttgagcagccctcgctggaacatgtcctgccaccaagtgccagctccctctctgtctgcaccagggagtagtacccccagctctgagaaagaggcggcatcccctaggccaagtggaaagaggctggggttcccatttgactccagtcccaggcagccatggggatctcgggtcagttccagccttcctctccaactcttcagccctgtgttctgctggggccatgaaggcagaaggtttagcctctgagaagccctcttcttcccccacccctttccaggagaaggggctgcccctccaagccctacttgtatgtgcggagtcacactgcagtgccgaacagtattagctcccgttcccaagtgtggactccagaggggctggaggcaagctatgaacttgctcgctggcccacccctaagactggtacccatttccttttcttaccctgatctccccagaagcctcttgtggtggtggctgtgccccctatgccctgtggcatttctgcgtcttactggcaaccacacaactcagggaaaggaatgcctgggagtgggggtgcaggcgggcagcactgagggaccctgccccgcccctccccccaggcccctttcccctgcagcttctcaagtgagactgacctgtctcacccagcagccactgcccagccgcactccaggcaagggccagtgcgcctgctcctgaccactgcaatcccagcgcccaaggaaggccacttctcaactggcagaacttctgaagtttagaattggaattacttccttactagtgtcttttggcttaaattttgtcttttgaagttgaatgcttaatcccgggaaagaggaacaggagtgccagactcctggtctttccagtttagaaaaggctctgtgccaaggagggaccacaggagctgggacctgcctgcccctgtcctttccccttggttttgtgttacaagagttgttggagacagtttcagatgattatttaatttgtaaatattgtacaaattttaatagcttaaattgtatatacagccaaataaaaacttgcattaacaaaaaaaaaaaaaaaaa

SEQ ID NO: 2 - protein, 617 aa, Homo sapiens [sequence omitted]

SEQ ID NO: 3 - DNA, 3028 bp, Homo sapiens
  CDS (60)..(1910)
gcagtcgcgcggattgagcgggctcgcggcgctgggttcctggtctccgggccagggcaatgttccgcacggcagtgatgatggcggccagcctggcgctgaccggggctgtggtggctcacgcctactacctcaaacaccagttctaccccactgtggtgtacctgaccaagtccagccccagcatggcagtcctgtacatccaggcctttgtccttgtcttccttctgggcaaggtgatgggcaaggtgttctttgggcaactgagggcagcagagatggagcaccttctggaacgttcctggtacgccgtcacagagacttgtctggccttcaccgtttttcgggatgacttcagcccccgctttgttgcactcttcactcttcttctcttcctcaaatgtttccactggctggctgaggaccgtgtggactttatggaacgcagccccaacatctcctggctctttcactgccgcattgtctctcttatgttcctcctgggcatcctggacttcctcttcgtcagccacgcctatcacagcatcctgacccgtggggcctctgtgcagctggtgtttggctttgagtatgccatcctgatgacgatggtgctcaccatcttcatcaagtatgtgctgcactccgtggacctccagagtgagaacccctgggacaacaaggctgtgtacatgctctacacagagctgtttacaggcttcatcaaggttctgctgtacatggccttcatgaccatcatgatcaaggtgcacaccttcccactctttgccatccggcccatgtacctggccatgagacagttcaagaaagctgtgacagatgccatcatgtctcgccgagccatccgcaacatgaacaccctgtatccagatgccaccccagaggagctccaggcaatggacaatgtctgcatcatctgccgagaagagatggtgactggtgccaagagactgccctgcaaccacattttccataccagctgcctgcgctcctggttccagcggcagcagacctgccccacctgccgtatggatgtccttcgtgcatcgctgccagcgcagtcaccaccacccccggagcctgcggatcaggggccaccccctgccccccaccccccaccactcttgcctcagccccccaacttcccccagggcctcctgcctccttttcctccaggcatgttcccactgtggccccccatgggcccctttccacctgtcccgcctccccccagctcaggagaggctgtggctcctccatccaccagtgcagccctttctcggcccagtggagcagctacaaccacagctgctggcaccagtgctactgctgcttctgccacagcatctggcccaggctctggctctgccccagaggctggccctgcccctggtttccccttccctcctccctggatgggtatgcccctgcctccaccctttgccttccccccaatgcctgtgccccctgcgggctttgctgggctgaccccagaggagctacgagctctggagggccatgagcggcagcacctggaggcccggctgcagagcctgcgtaacatccacacactgctggacgccgccatgctgcagatcaaccagtacctcaccgtgctggcctccttggggcccccccggcctgccacttcagtcaactccactgaggggactgccactacagttgttgctgctgcctcctccaccagcatccctagctcagaggccacgaccccaaccccaggagcctccccaccagcccctgaaatggaaaggcctccagctcctgagtcagtgggcacagaggagatgcctgaggatggagagcccgatgcagcagagctccgccggcgccgcctgcagaagctggagtctcctgttgcccactgacactgccccagcccagccccagcctctgctcttttgagcagccctcgctggaacatgtcctgccaccaagtgccagctccctctctgtctgcaccagggagtagtacccccagctctgagaaagaggcggcatcccctaggccaagtggaaagaggctggggttcccatttgactccagtcccaggcagccatggggatctcgggtcagttccagccttcctctccaactcttcagccctgtgttctgctggggccatgaaggcagaaggtttagcctctgagaagccctcttcttcccccacccctttccaggagaaggggctgcccctccaagccctacttgtatgtgcggagtcacactgcagtgccgaacagtattagctcccgttcccaagtgtggactccagaggggctggaggcaagctatgaacttgctcgctggcccacccctaagactggtacccatttccttttcttaccctgatctccccagaagcctcttgtggtggtggctgtgccccctatgccctgtggcatttctgcgtcttactggcaaccacacaactcagggaaaggaatgcctgggagtgggggtgcaggcgggcagcactgagggaccctgccccgcccctccccccaggcccctttcccctgcagcttctcaagtgagactgacctgtctcacccagcagccactgcccagccgcactccaggcaagggccagtgcgcctgctcctgaccactgcaatcccagcgcccaaggaaggccacttctcaactggcagaacttctgaagtttagaattggaattacttccttactagtgtcttttggcttaaattttgtcttttgaagttgaatgcttaatcccgggaaagaggaacaggagtgccagactcctggtctttccagtttagaaaaggctctgtgccaaggagggaccacaggagctgggacctgcctgcccctgtcctttccccttggttttgtgttacaagagttgttggagacagtttcagatgattatttaatttgtaaatattgtacaaattttaatagcttaaattgtatatacagccaaataaaaacttgcattaacaaaaaaaaaaaaaaaaa

SEQ ID NO: 4 - protein, 616 aa, Homo sapiens
MFRTAVMMAASLALTGAVVAHAYYLKHQFYPTVVYLTKSSPSMAVLYIQAFVLVFLLGKVMGKVFFGQLRAAEMEHLLERSWYAVTETCLAFTVFRDDFSPRFVALFTLLLFLKCFHWLAEDRVDFMERSPNISWLFHCRIVSLMFLLGILDFLFVSHAYHSILTRGASVQLVFGFEYAILMTMVLTIFIKYVLHSVDLQSENPWDNKAVYMLYTELFTGFIKVLLYMAFMTIMIKVHTFPLFAIRPMYLAMRQFKKAVTDAIMSRRAIRNMNTLYPDATPEELQAMDNVCIICREEMVTGAKRLPCNHIFHTSCLRSWFQRQQTCPTCRMDVLRASLPAQSPPPPEPADQGPPPAPHPPPLLPQPPNFPQGLLPPFPPGMFPLWPPMGPFPPVPPPPSSGEAVAPPSTSAALSRPSGAATTTAAGTSATAASATASGPGSGSAPEAGPAPGFPFPPPWMGMPLPPPFAFPPMPVPPAGFAGLTPEELRALEGHERQHLEARLQSLRNIHTLLDAAMLQINQYLTVLASLGPPRPATSVNSTEGTATTVVAAASSTSIPSSEATTPTPGASPPAPEMERPPAPESVGTEEMPEDGEPDAAELRRRRLQKLESPVAH